(19) United States
(12) Patent Application Publication
(10) Pub. No.: US 2006/0142287 A1
(43) Pub. Date: Jun. 29, 2006

(54) Use of a combination of dppe with other chemotherapeutic agents for the treatment of breast cancer

(76) Inventor: Lorne J. Brandes, Winnipeg (CA)

Correspondence Address: ROTHWELL, FIGG, ERNST & MANBECK, P.C., 1425 K STREET, N.W., SUITE 800, WASHINGTON, DC, 20005, US

(21) Appl. No.: 10/527,686

(22) PCT Filed: Sep. 5, 2003
(86) PCT No.: PCT/CA03/01343

§ 371 Date: Oct. 7, 2005

Related U.S. Application Data
(60) Provisional application No. 60/409,584, filed on Sep. 11, 2002.

(51) Int. Cl. A61K 31/537; A61K 31/495; A61K 31/138

(52) U.S. Cl. 514/238.8; 514/252.12; 514/651

(57) ABSTRACT

An improved adjuvant treatment of stage I or II breast cancer in which patients are administered a chemotherapeutic agent active in breast cancer is provided in which a diphenyl compound which is a potent antagonist of histamine binding at the intracellular histamine receptor is initially administered prior to administration of the chemotherapeutic agent. Such pretreatment is expected to lead to longer overall survival for patients who have received no prior chemotherapy or no prior treatment type (chemotherapy, radiotherapy and/or hormone treatment) or estrogen receptor-negative tumors.

### FIELD OF THE INVENTION

The present invention relates to the adjuvant treatment of stage I or II breast cancer.

### BACKGROUND OF THE INVENTION

Newly-diagnosed patients with breast cancer normally undergo systemic therapy, after surgery to remove the tumor, if cancer cells have spread to the regional lymph nodes in the axilla (Stage II). In such cases, especially in women with tumors that are not estrogen responsive (estrogen receptor-negative tumors), adjuvant chemotherapy is prescribed. Adjuvant chemotherapy is also prescribed for newly-diagnosed patients with breast cancer, after surgery to remove the tumor, when the cancer cells have not spread to the regional lymph nodes in the axilla (Stage I), but the tumor cells are estrogen receptor-negative. Historically, adjuvant chemotherapy, given for 4 or 6 cycles, has consisted of drugs active against breast cancer. These agents include anthracyclines (doxorubicin or epirubicin) and taxanes (Taxol, a trademark of Bristol-Myers Squibb for paclitaxel) or Taxotere (a trademark of Aventis Pharma for docetaxel). An overall decrease of about 10% in the risk of recurrence has been achieved with this approach.

The objective of chemotherapy is the total extermination of clonogenic tumor or malignant cells, with minimal damage to the patient. However, one of the major limitations of the chemotherapeutic approach for managing human cancer is the general inability of anticancer drugs to discriminate between normal and tumorous cells. Anti-neoplastic agents have the lowest therapeutic indicies of any class of drugs used in humans and hence produce significant and potentially life-threatening toxicities. Certain commonly-used anti-neoplastic agents have unique and acute toxicities for specific tissues. For example, the vinca alkaloids possess significant toxicity for nervous tissues, while adriamycin has specific toxicity for heart tissue and bleomycin has for lung tissue. In general, almost all members of the major categories of anti-neoplastic agents have considerable toxicities for normal cells of gastrointestinal, epidermal and myelopoietic tissues.

Generally, the dose-limiting consideration for chemical management of cancer in humans is the toxicity that anti-neoplastic agents have for the pluripotent stem cells of myelopoietic tissue. This toxicity arises from the fact that most anticancer drugs function preferentially against proliferating cells but with no significant capacity to discriminate between cycling normal and cycling tumor tissues.

In U.S. Pat. Nos. 6,288,799, 5,859,065, 5,708,329, 5,747,543 and 5,618,846, all assigned to University of Manitoba and the disclosures of which are incorporated herein by reference, there is described an improved method for the in vivo chemotherapeutic treatment of cancer in which there is first administered a compound which inhibits normal cell proliferation while promoting malignant cell proliferation, specifically a potent antagonist selective for intracellular histamine receptors, in an amount sufficient to inhibit the binding of intracellular histamine to the receptors in normal and malignant cells. Following sufficient time to permit the inhibition of binding of intracellular histamine, a chemotherapeutic agent is administered. An enhanced toxic effect on the cancer cells from the chemotherapeutic agent is obtained while any adverse effect of the chemotherapeutic agent on normal cells, particularly bone marrow and gastro-intestinal cells, is significantly ameliorated. One useful compound which is inhibits normal cell proliferation while promoting malignant cell proliferation is N,N-diethyl-2-[4-(phenylmethyl)-phenoxy]ethanamine, abbreviated herein as DPPE.

### SUMMARY OF INVENTION

It has now surprisingly been found, in a comparison of DPPE/doxorubicin with doxorubicin alone in a Phase III clinical trial, that a significant (200 to 300%) increase in overall survival occurred in patients with metastatic or recurrent breast cancer and who had no previous chemotherapy or no previous treatment including chemotherapy, radiotherapy and/or hormone treatment.

This observation is suggestive that such adjuvant chemotherapy of patients with stage I or II breast cancer by a combination of DPPE and doxorubicin also leads to a significant increase in overall survival compared to the current approach. In the present invention, patients with stage I or II breast cancer are subjected to chemotherapy, post surgery, including pretreatment with DPPE and related compounds followed by treatment with doxorubicin, epirubicin or other anthracycline chemotherapeutic agent active in breast cancer, optionally in combination with a taxane chemotherapeutic agent active in human cancer.

Accordingly, in one aspect, the present invention provides a method of adjuvant chemotherapy in human patients with stage I or II breast cancer, which comprises, following surgical removal of the tumor:

(a) first administering to said patients at least one diphenyl compound of the formula: 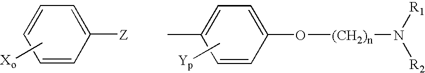
wherein X and Y are each fluorine, chlorine or bromine, Z is an alkylene group of 1 to 3 carbon atoms or ═C═O, or the phenyl groups are joined to form a tricyclic ring, o and p are 0 or 1, R₁ and R₂ are each an alkyl group containing 1 to 3 carbon atoms or are joined together to form a heterocyclic ring with the nitrogen atom and n is 1, 2 or 3, or pharmaceutically-acceptable salts thereof, and

(b) following sufficient time to permit inhibition of binding of intracellular histamine, subsequently administering to the patient a chemotherapeutic agent active in breast cancer.

In the application of the present invention, the diphenyl compound and the chemotherapeutic agent are generally administered by intravenous infusion. In one preferred procedure, a solution of the diphenyl compound is administered to the patient over a desired period of time prior to administration of the chemotherapeutic agent and a solution of the chemotherapeutic agent in combination with the diphenyl compound then is administered for the period of administration of the chemotherapeutic agent. If desired, a solution of the diphenyl compound is administered after completion of the administration of the chemotherapeutic agent for a desired period of time to ameliorate side effects from the chemotherapeutic agent administration.

### BRIEF DESCRIPTION OF DRAWINGS

FIG. 1 is a graphical representation of results of treatment with a combination of DPPE/DOX in comparison to doxorubicin alone (solid line, DPPE/DOX; dotted line, DOX) in the human Phase III clinical trial outlined below and depicts the survival by duration for patients with metastatic and/or recurrent breast cancer and who have had no prior chemotherapy;

FIG. 2 is a graphical representation of results of treatment with a combination of DPPE/DOX in comparison with doxorubicin alone (solid line, DPPE/DOX; dotted line, DOX) in the human Phase III clinical trial outlined below and depicts the survival by duration for patients with metastatic and/or recurrent breast cancer and who have had no previous treatment type; and

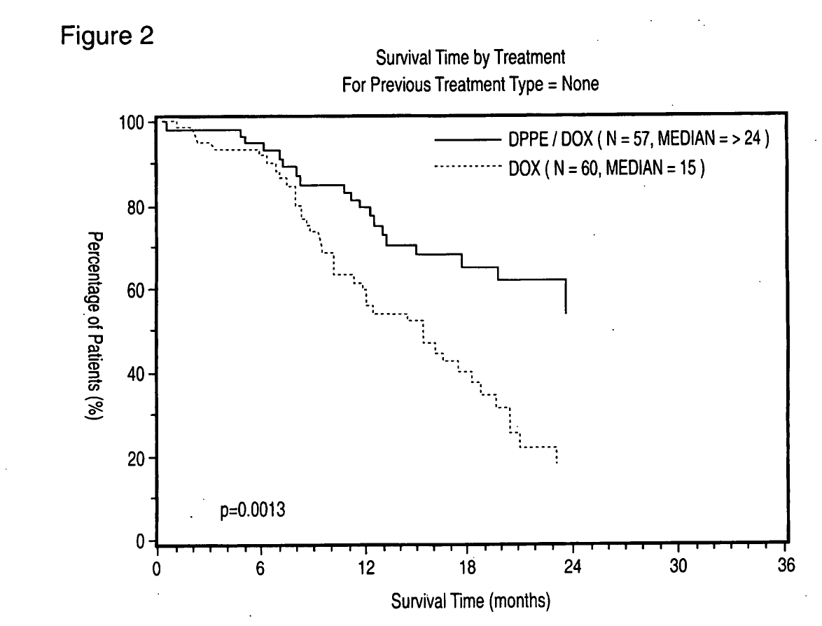

FIG. 3 is graphical representation of results of treatment with a combination of DPPE/DOX in comparison with doxorubicin alone (solid line, DPPE/DOX; dotted line, DOX) in the human Phase III clinical trial outlined below and depicts the survival by duration for patients with metastatic and/or recurrent breast cancer whose tumors were estrogen receptor (ER) negative.

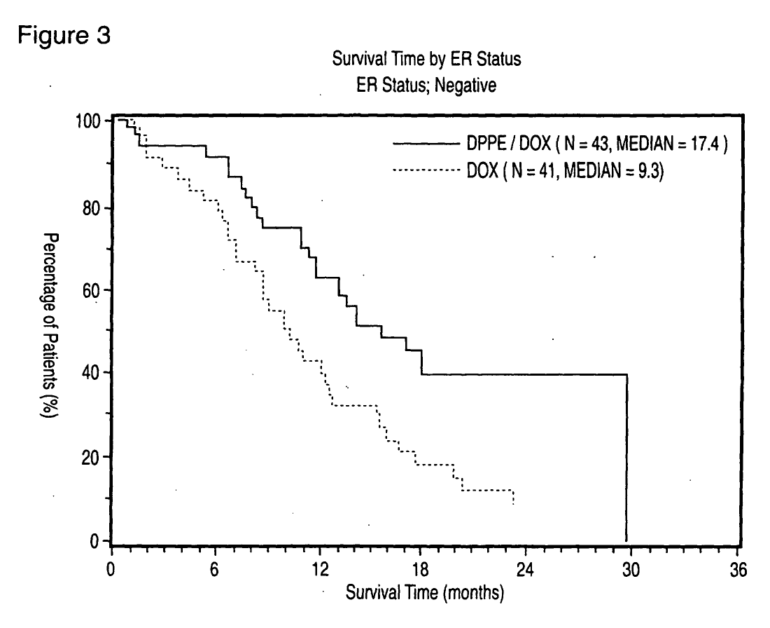

### GENERAL DESCRIPTION OF INVENTION

in the present invention, a diphenyl compound is used which is a potent antagonist of histamine binding at the intracellular histamine receptor and is administered in an amount sufficient to inhibit the binding of intracellular histamine at the intracellular binding site (H_{IC}) in normal cells. Such compounds exhibit a pKi of at least about 5, preferably at least about 5.5.

Specific potent compounds which are useful in the present invention are diphenyl compounds of the formula: 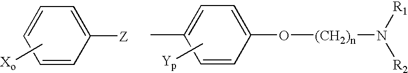
wherein X and Y are each fluorine, chlorine or bromine, Z is an alkylene group of 1 to 3 carbon atoms or ═C═O, o and p are 0 or 1, R₁ and R₂ are each alkyl groups containing 1 to 3 carbon atoms or are joined together to form a hetero-ring with the nitrogen atom and n is 1, 2 or 3. Pharmaceutically-acceptable salts of the diphenyl compounds may be employed.

Alternatively, the benzene rings may be joined to form a tricyclic ring, in accordance with the structure:

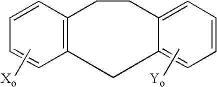

In one preferred embodiment, the group 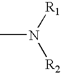
is a diethylamino group, although other alkylamino groups may be employed, such as dimethylamino, and, in another preferred embodiment, a morpholino group, although other heterocyclic ring groups may be employed, such as piperazino. o and p are usually 0 when Z is an alkylene group and n may be 2. In one particularly preferred embodiment, Z is —CH₂—, n is 2, o and p are each 0 and 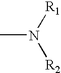
is a diethylamino group. This compound, namely N,N-diethyl-2-[4-(phenylmethyl)-phenoxy]ethanamine, in the form of the free base or in the form of its hydrochloride or other pharmaceutically-acceptable salt, is abbreviated herein as DPPE. In addition to a methyl group linking the benzene rings, other linking groups may be employed, such as ═C═O. Other substitutents may be provided on the benzene rings in addition to the halogen atoms, for example, an imidazole group.

The chemotherapeutic agents employed herein is one which is active in breast cancer. Such chemotherapeutic agents active in breast cancer include anthracyclines, such as doxorubicin and epirubicin; anthracene diones, such as mitoxantrone; and taxanes, such as Taxol (a trademark of Bristol-Myers Squibb for paclitaxel) or Taxotere (a trademark of Aventis Pharma for docetaxel). The chemotherapeutic agent, or a mixture of such agents, is administered in any manner consistent with its normal manner of administration in conventional breast cancer therapy, namely by intravenous infusion of a solution thereof. Specific combinations of chemotherapeutic agents which may be used in the procedures of the present invention include doxorubicin or epirubicin with Taxol or Taxotere.

The administration of the diphenyl compound to the patient prior to administration of the chemotherapeutic agent is necessary in order to permit the diphenyl compound to inhibit the binding of intracellular histamine in normal and malignant cells and thereby, in effect, shut down the proliferation of the normal cells, but increase proliferation of malignant cells.

The length of time prior to administration of the chemotherapeutic agent(s) that the diphenyl compound is administered depends on the diphenyl compound, its mode of administration and the size of the patient. Generally, the diphenyl compound is administered to the patient for about 30 to about 90 minutes, preferably about 60 minutes, prior to administration of the chemotherapeutic agent(s).

The quantity of diphenyl compound administered to the patient depends on the side effects to be ameliorated, but should be at least sufficient to inhibit binding of intracellular histamine in normal cells. The quantity required to achieve the beneficial effects of the present invention depends upon the diphenyl compound employed, the chemotherapeutic agent(s) employed and the quantity of such agent(s) employed.

In general, the quantity of diphenyl compound employed in humans is from about 8 to about 320 mg/M² of human to which the diphenyl compound is administered, with about 8 and 240 mg/M² being the optimal dose for gastro-intestinal and bone marrow protection, respectively. Over this dose range, the present invention is able to achieve an enhanced chemotherapeutic effect of chemotherapeutic agent on breast cancer cells while, at the same time, also protecting normal cells form damage by the chemotherapeutic agent(s) in a wide variety of circumstances where traditional chemotherapy leads to damage of normal cells or tissues not involved in the disease process.

In the treatment of stage I or II breast cancer, the diphenyl compound preferably is used in an amount of about 3 to about 10 mg/kg of patient, administered intravenously over a period of about 30 to about 90 minutes prior to administration of the chemotherapeutic agent(s) and continuing for the period of administration of the chemotherapeutic agent(s). In the specific Phase III clinical trial described herein conducted on patients with metastatic and/or recurrent breast cancer, there was employed 5.3 mg/kg of DPPE in the form of the base (equivalent to 6 mg/kg of DPPE in the form of its hydrochloride), administered intravenously as an aqueous solution thereof over 80 minutes, with the last twenty minutes being accompanied by infusion of the specific chemotherapeutic agent.

The chemotherapy agent active in breast cancer which is employed herein preferably is used in a total amount of about 50 to about 75 mg/M² of patient for doxorubicin or epirubicin, about 175 to about 225 mg/M² for Taxol and about 75 to about 100 mg/M² for Taxotere. In the specific Phase III clinical trial described herein conducted on patients with metastatic and/or recurrent breast cancer, there was employed 60 mg/M² of doxorubicin administered over the last 20 minutes of infusion of the DPPE solution. However, epirubicin is equally potent and may be used in place of doxorubicin.

As set forth herein, a Phase III clinical trial was conducted on patients having metastatic and/or recurrent breast cancer in which one group of patients was administered DPPE followed by doxorubicin while a control group was administered doxorubicin alone. Various data from the clinical trial were collected and analyzed. Details of the clinical trial are set forth in Example 1 while the analysis of the data and comparison to studies not employing DPPE are set forth in Example 2.

As noted earlier, in the Phase III clinical trial reported herein, patients who had metastatic and/or recurrent breast cancer and who have had no prior chemotherapy or who had no prior treatment type or who had estrogen receptor-negative tumors, exhibited longer overall survival when pretreated with DPPE than those who did not have such pretreatment.

Accordingly, in another aspect of the invention, there is provided a method of achieving enhanced survival in human patients with stage I or II breast cancer, which comprises: (a) selecting for chemotherapy treatment patients who have had no prior chemotherapy treatment or any previous treatment type or estrogen receptor-negative tumors, and (b) subject said selected patients to chemotherapy treatment for a plurality of cycles at predetermined intervals, each said cycle comprising:

(i) first administering to said selected patients at least one diphenyl compound of the formula: 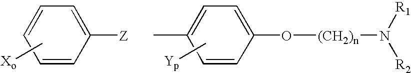
wherein X and Y are each fluorine, chlorine or bromine, Z is an alkylene group of 1 to 3 carbon atoms or ═C═O, or the phenyl groups are joined to form a tricyclic ring, o and p are 0 or 1, R₁ and R₂ are each an alkyl group containing 1 to 3 carbon atoms or are joined together to form a heterocyclic ring with the nitrogen atom and n is 1, 2 or 3, or pharmaceutically-acceptable salts thereof, and (ii) following sufficient time to permit inhibition of binding of intracellular histamine, subsequently administering to the patient a chemotherapeutic agent active in breast cancer.

The selected patients may be treated for about 4 to about 6 cycles at predetermined intervals of about 21 to about 28 days. In such procedure, the various alternatives, materials and doses discussed above may be used.

### EXAMPLES

### Example 1

This Example describes a Phase III clinical trial of the treatment of patients and metastatic and/or recurrent breast cancer.

Patients were treated with doxorubicin (DOX) alone or a combination of doxorubicin and DPPE. DPPE, in the free base form, was administered intravenously at a dose of 5.3 mg/kg over 80 minutes with doxorubicin administered at a dose of 60 mg/M² over the last 20 minutes while the control group received a dose of 60 mg/M² of doxorubicin alone. The patients were subjected to a number of cycles of chemotherapy, each followed by a 21 to 28 day rest period, until a cumulative dose of up to 450 mg/M² of doxorubicin had been administered to the patient.

305 patients participated in the study. 152 patients were randomized to DPPE/doxorubicin and 153 patients received doxorubicin alone. Median age was 53 years, 90% had received no prior chemotherapy for metastatic disease and 60% had viceral disease.

### Example 2

This Example analyzes the data obtained in the Phase III clinical trial described in Example 1.

The survival time for patients was determined for those patients with metastatic breast cancer receiving no prior chemotherapy treatment in relation to those who had received prior chemotherapy treatment, and for patients that had received no previous treatment type (namely, chemotherapy, radiotherapy and/or hormone treatment) and those who had received previous treatment. The survival times were determined for patients receiving the DPPE/DOX combination and DOX alone.

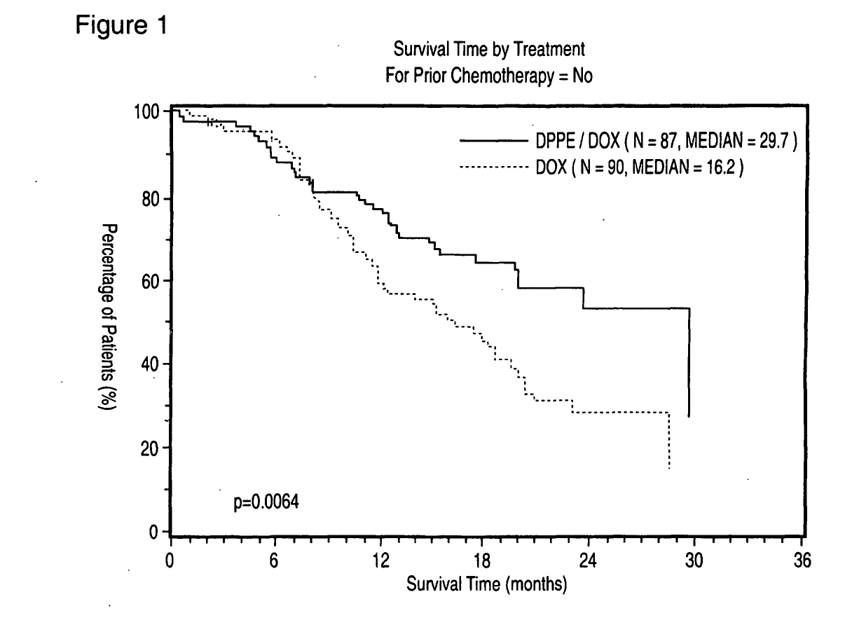

These results are shown in FIGS. 1 (no prior chemotherapy), 2 (no previous treatment type) and 3 (estrogen receptor-negative tumors). As may be seen in these Figures, a significant increase in median overall survival as between treatment with the DPPE/DOX combination and DOX alone was observed in patients with metastatic disease who had no previous chemotherapy, namely 29.7 months (N=87) for the DPPE/DOX combination in comparison to 16.2 months (N=90) (P=0.006) for DOX alone, or no previous treatment, namely greater than 24 months (N=57) for the DPPE/DOX combination in comparison to 15 months (N=60) (P=0.001) for DOX alone, or who had estrogen receptor-negative tumors, namely, 17.4 months (N=43) for the DPPE/DOX combination in comparison to 9.3 months (N=41) (P=0.009) for DOX alone.

These results are suggestive that an adjuvant treatment of patients with stage I or II breast cancer may lead to similar improvements in overall survival time.

### SUMMARY OF INVENTION

In summary of this disclosure, the present invention provides a method of achieving enhanced survival for patients with stage I or II breast cancer. Modifications are possible within the scope of the invention.

What I claim is:

1. A method of adjuvant chemotherapy in human patients with stage I or II breast cancer, which comprises, following surgical removal of tumor:
(a) first administering to said patients at least one diphenyl compound of the formula: 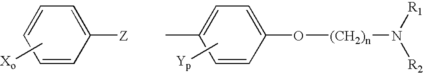
wherein X and Y are each fluorine, chlorine or bromine, Z is an alkylene group of 1 to 3 carbon atoms or ═C═O, or the phenyl groups are joined to form a tricyclic ring, o and p are 0 or 1, R₁ and R₂ are an alkyl each group containing 1 to 3 carbon atoms or are joined together to form a heterocyclic ring with the nitrogen atom and n is 1, 2 or 3, or pharmaceutically-acceptable salts thereof, and
(b) following sufficient time to permit inhibition of binding of intracellular histamine, subsequently administering to the patient a chemotherapeutic agent active in breast cancer.

2. The method of claim 1 wherein the group 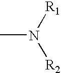
is a diethylamino group, a dimethylamino group, a morpholino group or a piperazino group.

3. The method of claim 1 wherein the group 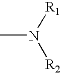
is a diethylamino group, Z is —CH₂, n is 2 and o and p are each 0.

4. The method of claim 3 wherein diphenyl compound is in the form of a hydrochloride salt or free base.

5. The method of claim 1 wherein said chemotherapeutic agent active in breast cancer is doxorubicin.

6. The method of claim 4 wherein said chemotherapeutic agent active in breast cancer is doxorubicin or epirubicin alone or in combination with taxanes (Taxol or Taxotere).

7. The method of claim 1 wherein said diphenyl compound is administered to the patient about 30 to about 90 minutes prior to said administration of said chemotherapeutic agent.

8. The method of claim 7 wherein said time is about 60 minutes.

9. The method of claim 6 wherein said diphenyl compound is administered by intravenous infusion of a solution thereof over a period of time of up to about 90 minutes prior to administration of said chemotherapeutic agent and is maintained during administration of said chemotherapeutic agent.

10. The method of claim 9 wherein said diphenyl compound is administered for about 60 minutes prior to administration of said chemotherapeutic agent and is maintained during about 20 minutes intravenous infusion of said chemotherapeutic agent.

11. The method of claim 7 wherein said diphenyl compound is administered in an amount of about 8 to about 240 mg/M² of said patient.

12. The method of claim 11 wherein said amount is about 3 to about 10 mg/kg of patient.

13. The method of claim 9 wherein said diphenyl compound is administered in an amount of about 3 to about 10 mg/kg of patient.

14. The method of claim 10 wherein said diphenyl compound is administered in an amount of about 6 mg/kg in the form of the hydrochloride salt or 5.3 mg/kg in the form of the free base.

15. The method of claim 11 wherein said chemotherapeutic agent is administered in an amount of about 50 to about 75 mg/M² of patient for doxorubicin or epirubicin, about 175 to about 225 mg/M² for Taxol and about 75 to about 100 mg/M² for Taxotere.

16. The method of claim 14 wherein said chemotherapeutic agent is administered in an amount of about 60 mg/M² of patient.

17. The method of claim 1 wherein said patients with stage I or II breast cancer are patients who have received no prior chemotherapy treatment.

18. The method of claim 1 wherein said patients with stage I or II breast cancer are patients who have received no prior treatment type.

19. The method of claim 1 wherein the human patients with stage I or II breast cancer are patients with estrogen receptor-negative tumors.

20. A method of achieving enhanced survival in human patients with stage I or II breast cancer, which comprises:
(a) selecting for chemotherapy treatment patients who have had no prior chemotherapy treatment or any previous treatment type or estrogen receptor-negative tumors, and
(b) subject said selected patients to chemotherapy treatment for a plurality of cycles at predetermined intervals, each said cycle comprising:
(i) first administering to said selected patients at least one diphenyl compound of the formula: 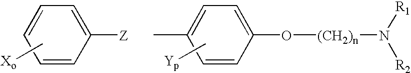
wherein X and Y are each fluorine, chlorine or bromine, Z is an alkylene group of 1 to 3 carbon atoms or ═C═O, or the phenyl groups are joined to form a tricyclic ring, o and p are 0 or 1, R₁ and R₂ are each an alkyl group containing 1 to 3 carbon atoms or are joined together to form a heterocyclic ring with the nitrogen atom and n is 1, 2 or 3, or pharmaceutically-acceptable salts thereof, and
(ii) following sufficient time to permit inhibition of binding of intracellular histamine, subsequently administering to the patient a chemotherapeutic agent active in breast cancer.

21. The method claimed in claim 20, wherein said selected patients are treated for about 4 to about 6 cycles and predetermined intervals of about 21 to about 28 days.
